(19) United States
(12) Patent Application Publication
(10) Pub. No.: US 2024/0139348 A1
(43) Pub. Date: May 2, 2024

(54) MULTI-MODALITY MOLECULAR IMAGING PROBE, AND PREPARATION METHOD AND USE THEREOF

(71) Applicants: WENZHOU MEDICAL UNIVERSITY, Wenzhou City (CN); Lanzhou University, Lanzhou City (CN)

(72) Inventors: Jiaxi Ru, Wenzhou City (CN); Xiaoliang Tang, Lanzhou City (CN); Xiufeng Huang, Wenzhou City (CN); Weisheng Liu, Lanzhou City (CN); Chao Liang, Wenzhou City (CN); Lingling Kang, Wenzhou City (CN); Xiaofen Chen, Lanzhou City (CN); Xian Shen, Wenzhou City (CN)

(21) Appl. No.: 18/465,807

(22) Filed: Sep. 12, 2023

(30) Foreign Application Priority Data
Sep. 22, 2022 (CN) 202211155484.8

(51) Int. Cl. A61K 49/10; A61B 5/00; G01N 1/30

(52) U.S. Cl. CPC A61K 49/103; A61B 5/0035; G01N 1/30; G01N 2001/302

(57) ABSTRACT

Disclosed are a multi-modality molecular imaging probe, and a preparation method and use thereof. The multi-modality molecular imaging probe has an ABA structure, with a magnetic functional unit of a gadolinium complex at the center, and two identical phosphorescent functional units of an iridium complex, which are reasonably integrated into the same one complex molecule. The multi-modality molecular imaging probe simultaneously introduces two optical functional units of the iridium complex and one magnetic functional unit of a gadolinium chelate in the same one complex molecule, which exhibits magnetic-optical dual functional properties. It therefore could be used to prepare both a contrast agent for magnetic resonance imaging and an optical probe for optical imaging.

### CROSS REFERENCE TO RELATED APPLICATION

This patent application claims the benefit and priority of Chinese Patent Application No. 202211155484.8, entitled “Multi-modality molecular imaging probe, and preparation method and use thereof” filed on Sep. 22, 2022, the disclosure of which is incorporated by reference herein in its entirety as part of the present application.

### TECHNICAL FIELD

The disclosure belongs to the field of coordination chemistry technology, and specifically relates to a multi-modality molecular imaging probe, and a preparation method and use thereof.

### BACKGROUND ART

Molecular imaging technology can provide visual and quantitative structural and functional information for a series of pathological and physiological changes such as the cause, occurrence, and development of cancer at the physiological and biochemical levels. At present, common molecular imaging technologies include X-ray, computed tomography (CT), ultrasound (US) imaging, optical imaging (OI), and magnetic resonance imaging (MRI). Each imaging technology has its own advantages and disadvantages. A single imaging modality no longer provides sufficient information for clinical diagnosis. The comprehensive application of multiple imaging methods will inevitably result in more comprehensive imaging results, thereby better understanding the biological mechanisms of diseases. Therefore, through the development of multi-modality molecular imaging probes and the comprehensive application of multiple imaging technologies, the advantages and disadvantages of different imaging methods are complemented, which will thereby provide more comprehensive and in-depth clinical diagnostic information for precise treatment of diseases.

In recent years, magnetic-optical dual modality molecular imaging probes have become a research hotspot in molecular imaging. Optical imaging technology could achieve real-time qualitative and quantitative testing of molecular level changes in physiological processes by visualizing and observing microscopic biological samples such as cells and tissue pathological sections. It has many advantages such as high sensitivity and good selectivity, and has become one of the internationally recognized mainstream research methods for studying molecular events in vivo. However, optical imaging also has the problem of insufficient optical penetration depth, which makes it difficult to achieve deep imaging of large animal models and human. Magnetic resonance imaging (MRI) can provide comprehensive and multi-angle integrated tomography scanning imaging of large living samples, allowing for more accurate imaging of various anatomically changed living organs and tissues. However, magnetic resonance imaging also has shortcomings such as insufficient spatial resolution, low sensitivity, and long imaging time. It can be seen that there is a good complementarity between the advantages and disadvantages of optical imaging and magnetic resonance imaging. The magnetic-optical dual modality molecular probe could take advantage of the complementarity between the two imaging methods, fully exert the advantages of magnetic resonance imaging (MRI), i.e., without limitation on structure penetration depth, and high sensitivity of optical imaging, and simultaneously acquire anatomical information and molecular functional information, which achieves diagnostic tasks difficult by a single imaging method, thereby providing more comprehensive imaging information. However, currently, molecular imaging probes disclosed in existing technologies either have the characteristics of magnetic resonance imaging or optical imaging, and result in single imaging, which limits their application.

### SUMMARY

In view of this, the present disclosure provides a multi-modality molecular imaging probe, and a preparation method and use thereof. The multi-modality molecular imaging probe according to the present disclosure includes both an iridium(III) complex with optical properties and a gadolinium(III) complex with magnetic properties.

In order to achieve the above objects, the present disclosure provides a multi-modality molecular imaging probe having a structure represented by formula I,

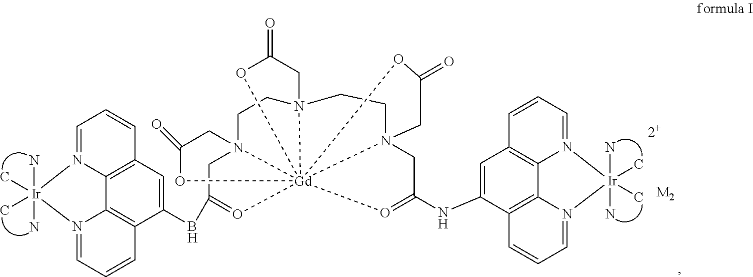

wherein in formula I, M comprises Cl⁻; and
each

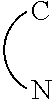

moiety is one selected from the group consisting of

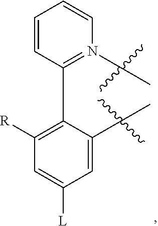

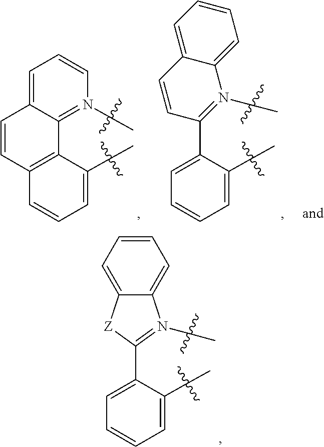

wherein
R and L are each independently selected from the group consisting of a halogen, an alkyl, and hydrogen; and
Z is selected from the group consisting of —O— and —S—.

In some embodiments, M is replaced with one selected from the group consisting of Br⁻, I⁻, NO₃⁻, and PF₆⁻.

The present disclosure further provides a method for preparing the multi-modality molecular imaging probe as described in above technical solutions, comprising steps of
dissolving an inorganic gadolinium(III) salt and a diethylenetriaminepentaacetic acid derivative, and performing first coordination reaction, to obtain a gadolinium(III) complex; and
dissolving the gadolinium(III) complex and an iridium(III) complex precursor, and performing second coordination reaction, to obtain the multi-modality molecular imaging probe;
wherein the diethylenetriaminepentaacetic acid derivative has a structure represented by formula I-1,

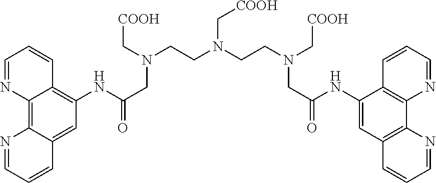 [SMILES: O=C(O)CN(CCN(CC(=O)O)CC(=O)Nc1cc2cccnc2c2ncccc12)CCN(CC(=O)O)CC(=O)Nc1cc2cccnc2c2ncccc12]

formula I-1; and
the iridium complex precursor has a structure represented by formula I-2,

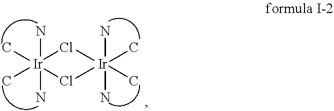

wherein in formula I-2, each

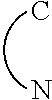

moiety is one selected from the group consisting of

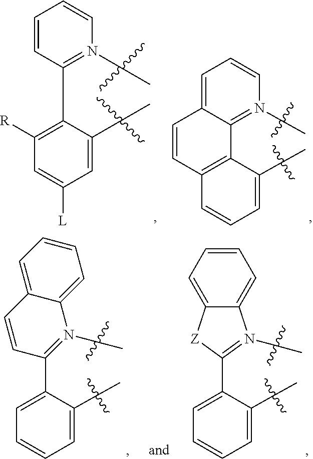

wherein R and L are each independently selected from the group consisting of a halogen, an alkyl, and hydrogen; and
Z is selected from the group consisting of —O— and —S—.

In some embodiments, the inorganic gadolinium salt comprises one or more selected from the group consisting of gadolinium nitrate, gadolinium chloride, and gadolinium perchlorate.

In some embodiments, a ratio of an amount in moles of the inorganic gadolinium salt to a sum of amounts in moles of the inorganic gadolinium salt and the diethylenetriaminepentaacetic acid derivative is in a range of (0.2-0.8):1.

In some embodiments, a ratio of an amount in moles of the gadolinium complex to a sum of amounts in moles of the gadolinium complex and the iridium complex precursor is in a range of (0.3-0.7):1.

In some embodiments, the first coordination reaction is performed at a temperature of 45-55° C. for 23-25 h.

In some embodiments, the second coordination reaction is performed at a temperature of 40-60° C. for 22-26 h.

In some embodiments, under the condition that M is replaced with one selected from the group consisting of Br⁻, I⁻, NO₃⁻, and PF₆⁻, replacing M with one selected from the group consisting of Br⁻, I⁻, NO₃⁻, and PF₆⁻ includes, after the second coordination reaction, subjecting a product obtained from the second coordination reaction to ion exchange reaction, wherein a reagent for the ion exchange reaction includes one selected from the group consisting of NaBr, NaI, NaNO₃, and ammonium hexafluorophosphate.

The present disclosure provides use of the multi-modality molecular imaging probe as described in above technical solutions or the multi-modality molecular imaging probe prepared by a method as described in above technical solutions in the preparation of a contrast agent for magnetic resonance imaging or the preparation of an optical probe for optical imaging.

The present disclosure provides a multi-modality molecular imaging probe having a structure represented by formula I. The multi-modality molecular imaging probe according to the present disclosure has an ABA structure, wherein B represents a magnetic functional unit of a gadolinium complex, and A represents a phosphorescence functional unit of an iridium complex, which are reasonably integrated into the same one complex molecule. This multi-modality molecular imaging probe introduces two optical functional units of an iridium complex and one magnetic functional unit of a gadolinium complex into the same one complex molecule, which exhibits magnetic-optical dual functional properties. It therefore could be used to prepare both a contrast agent for magnetic resonance imaging and an optical probe for optical imaging.

The present disclosure also provides a method for preparing the multi-modality molecular imaging probe as described above, including the following steps: dissolving an inorganic gadolinium salt and a diethylenetriaminepentaacetic acid derivative, and performing first coordination reaction, to obtain a gadolinium complex; and dissolving the gadolinium complex and an iridium complex precursor, and performing second coordination reaction, to obtain the multi-modality molecular imaging probe. In the present disclosure, a diethylenetriaminepentaacetic acid derivative is used as a raw material, and diethylenetriaminepentaacetic acid structure therein could bond with gadolinium ions to form a stable magnetic functional unit, and ortho phenanthroline structure therein could bond with an iridium complex precursor to form a phosphorescent functional unit, thereby achieving reasonable unity of optical and magnetic functions in a single complex molecule.

In the present disclosure, unless otherwise specified, the symbol

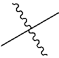

in structural formulas refers to the bonding site.

### BRIEF DESCRIPTION OF THE DRAWINGS

FIG. 1 shows an ultra violet (UV)-visible absorption spectrum of the multi-modality molecular imaging probe according to an embodiment of the present disclosure.

FIG. 2 shows a phosphorescence emission spectrum of the multi-modality molecular imaging probe according to an embodiment of the present disclosure.

FIG. 3A shows a confocal phosphorescence image of the multi-modality molecular imaging probe-treated EMT6 cells according to an embodiment of the present disclosure (referred to as Ir²Gd¹), in which emission was collected by the channel (580±20 nm) (λₑₓ=405 nm).

FIG. 3B shows a confocal fluorescence image of the multi-modality molecular imaging probe-treated EMT6 cells, then further incubated with mitochondrial red fluorescent probe (Mito-Tracker Red, MTR). The signal was collected by the channel (600±10 nm) (λₑₓ=561 nm).

FIG. 3C shows a confocal fluorescence image of the multi-modality molecular imaging probe-treated EMT6 cells, then further incubated with Lysosome green fluorescent probe (Lyso-Tracker Green, LTG). The signal was collected by the channel (510±10 nm) (λₑₓ=488 nm).

FIG. 3D shows a confocal bright-field image of the multi-modality molecular imaging probe-treated EMT6 cells.

FIG. 3E show a confocal fluorescence image of the multi-modality molecular imaging probe-treated EMT6 cells, then further incubated with a cell nucleus dye Hochest 33258 (i.e., H33258). The signal was collected by the channel (480±20 nm) (λₑₓ=405 nm).

FIG. 3F shows an overlay image of the bright-field, Ir²Gd¹, MTR, LTG, and Hochest 33258.

FIG. 3G shows degree of colocalization between Ir²Gd¹ and MTR.

FIG. 3H shows degree of colocalization between Ir²Gd¹ and LTG.

FIG. 3I shows degree of colocalization between Ir²Gd¹ and Hochest 33258.

FIG. 3J shows a confocal 2.5 D image of Ir²Gd¹.

FIG. 4A shows in vitro magnetic resonance imaging images of the multi-modality molecular imaging probe according to an embodiment of the present disclosure with different concentrations (i.e., 0, 0.025, 0.05, 0.1, 0.2, 0.4 mM).

FIG. 4B shows a relationship between T₁ weighted image and concentrations of the multi-modality molecular imaging probe.

FIG. 5 shows in vivo magnetic resonance imaging images by using the multi-modality molecular imaging probe according to an embodiment of the present disclosure.

FIG. 6 shows a structural formula of the multi-modality molecular imaging probe, which introduces two optical functional units of an iridium complex and one magnetic functional unit of a gadolinium complex into the same one complex molecule, which exhibits magnetic-optical dual functional properties.

### DETAILED DESCRIPTION OF THE EMBODIMENTS

The present disclosure provides a multi-modality molecular imaging probe having a structure represented by formula I,

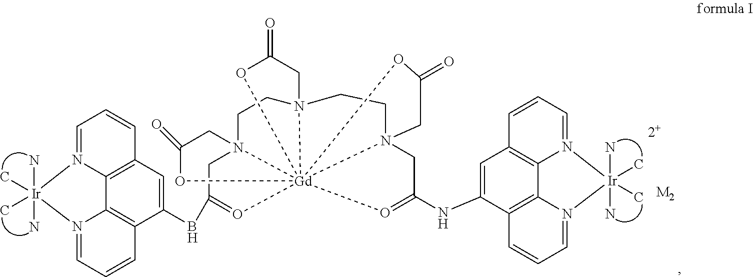

wherein in formula I, M comprises Cl⁻; and
each

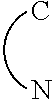

moiety is one selected from the group consisting of

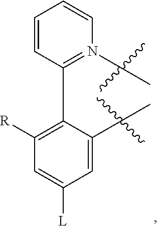

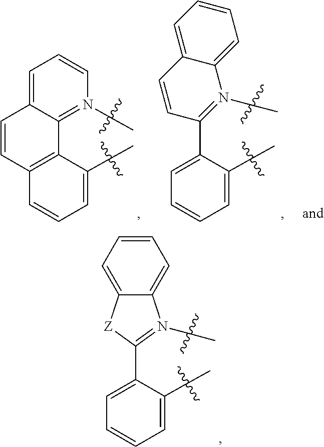

wherein
R and L are each independently selected from the group consisting of a halogen, an alkyl, and hydrogen; and
Z is selected from the group consisting of —O— and —S—.

In some embodiments, M in formula I is replaced with one selected from the group consisting of Br⁻, I⁻, NO₃⁻, and PF₆⁻, preferably PF₆⁻.

In some embodiments, the multi-modality molecular imaging probe has a structure selected from the group consisting of

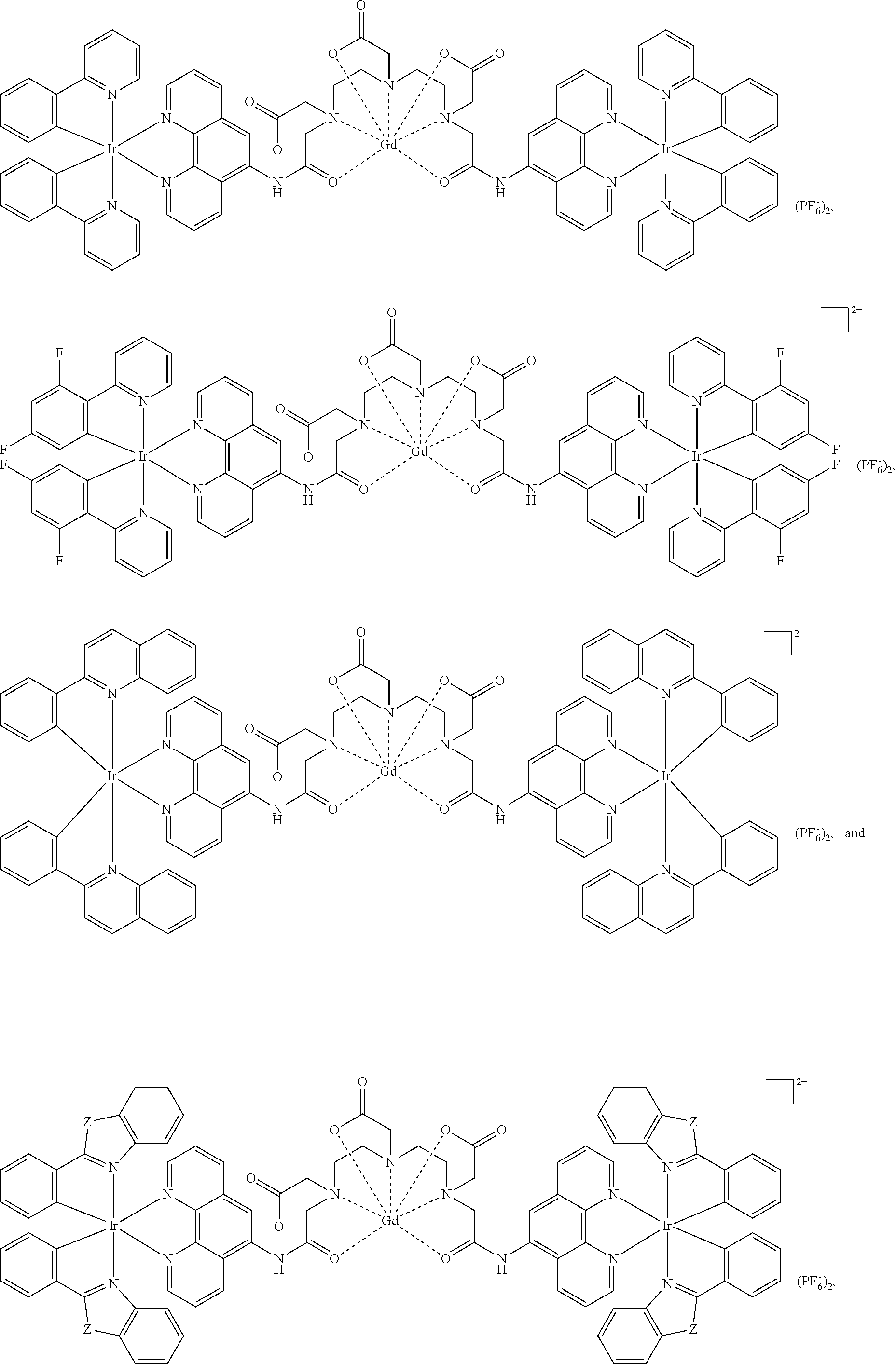

wherein Z is selected from the group consisting of —O— and —S—.

The present disclosure further provides a method for preparing the multi-modality molecular imaging probe as described in the above technical solutions, comprising steps of
dissolving an inorganic gadolinium salt and a diethylenetriaminepentaacetic acid derivative, and performing first coordination reaction, to obtain a gadolinium complex; and
dissolving the gadolinium complex and an iridium complex precursor, and performing second coordination reaction, to obtain the multi-modality molecular imaging probe;
wherein the diethylenetriaminepentaacetic acid derivative has a structure represented by formula I-1,

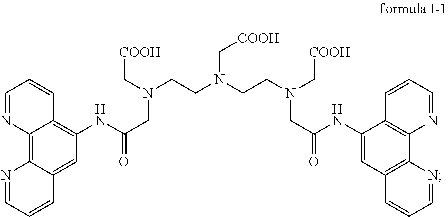 [SMILES: O=C(O)CN(CCN(CC(=O)O)CC(=O)Nc1cc2cccnc2c2ncccc12)CCN(CC(=O)O)CC(=O)Nc1cc2cccnc2c2ncccc12]

and
the iridium complex precursor has a structure represented by formula I-2,

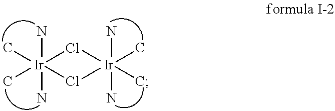

wherein in formula I-2, each

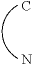

moiety is one selected from the group consisting of

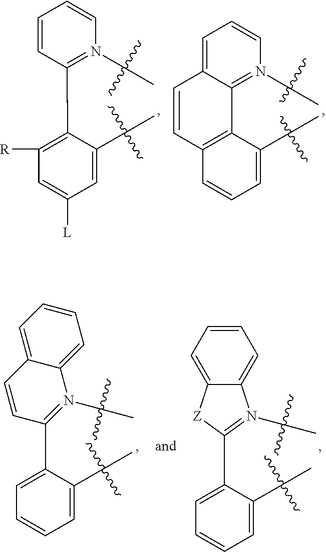

wherein R, L and Z each are defined the same as in formula I.

In the present disclosure, an inorganic gadolinium salt and diethylenetriaminepentaacetic acid derivative are dissolved, and subjected to first coordination reaction, to obtain a gadolinium complex.

In some embodiments of the disclosure, the inorganic gadolinium salt includes one or more selected from the group consisting of gadolinium nitrate, gadolinium chloride, and gadolinium perchlorate, and preferably gadolinium nitrate. In some embodiments, a ratio of an amount in moles of the inorganic gadolinium salt to a sum of amounts in moles of the inorganic gadolinium salt and the diethylenetriaminepentaacetic acid derivative is in a range of (0.2-0.8):1, and preferably 0.5:1.

In some embodiments of the present disclosure, a reagent for dissolving the inorganic gadolinium salt and the diethylenetriaminepentaacetic acid derivative includes an aqueous solution of methanol and/or an aqueous solution of ethanol. In some embodiments, a volume ratio of methanol to water in the aqueous solution of methanol is in a range of 1:5 to 5:1, and preferably 1:1 to 3:1. In some embodiments, a volume ratio of ethanol to water in the aqueous solution of ethanol is in a range of 1:5 to 5:1, and preferably 1:1 to 3:1.

In some embodiments of the present disclosure, the first coordination reaction is performed at a temperature of 45-55° C., and preferably 50° C. In some embodiments, the first coordination reaction is performed for 23-25 h, and preferably 24 hours. In some embodiments of the present disclosure, the first coordination reaction is carried out in an atmosphere of a protective gas, the protective gas including helium gas and/or nitrogen gas, and preferably being helium gas.

In the present disclosure, taking gadolinium nitrate as an example, the first coordination reaction is performed according to the reaction equation as follows:

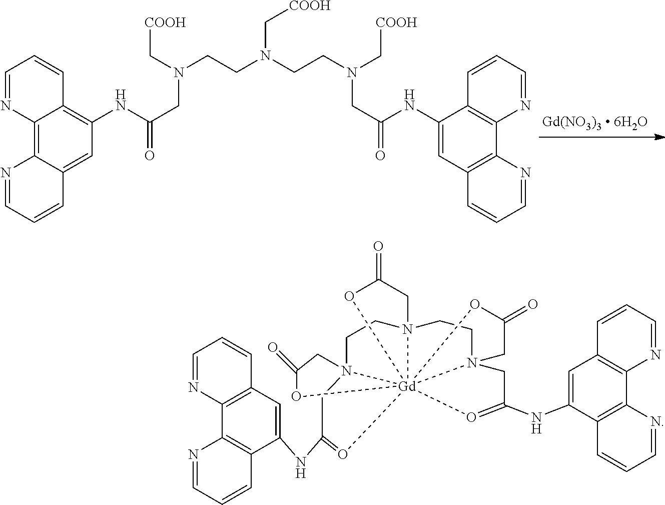

In some embodiments of the present disclosure, the method further includes, after the first coordination reaction, filtering a product obtained from the first coordination reaction. In the present disclosure, there is no specific limitation on the filtration, and operations well known to those skilled in the art may be adopted.

In the method according to the present disclosure, after obtaining the gadolinium complex, the gadolinium complex and the iridium complex precursor are dissolved, and subjected to second coordination reaction, to obtain the multi-modality molecular imaging probe.

In some embodiments of the present disclosure, a ratio of an amount in moles of the gadolinium complex to a sum of amounts in moles of the gadolinium complex and the iridium complex precursor is in a range of (0.3-0.7):1, and preferably 0.5:1.

In some embodiments of the disclosure, a reagent for dissolving the gadolinium complex and the iridium complex precursor is a mixed solvent of dichloromethane and methanol. In some embodiments, a volume ratio of dichloromethane to methanol in the mixed solvent is in a range of 1:(1-3), and preferably 1:2.

In some embodiments of the present disclosure, the second coordination reaction is performed at a temperature of 40-60° C., and preferably 50-55° C. In some embodiments, the second coordination reaction is performed for 22-26 hours, and preferably 24-25 hours. In some embodiments of the present disclosure, the second coordination reaction is carried out in an atmosphere of a protective gas, the protective gas including helium gas and/or nitrogen gas, and preferably being helium gas.

In the present disclosure, the second coordination reaction is performed according to the reaction equation as follows:

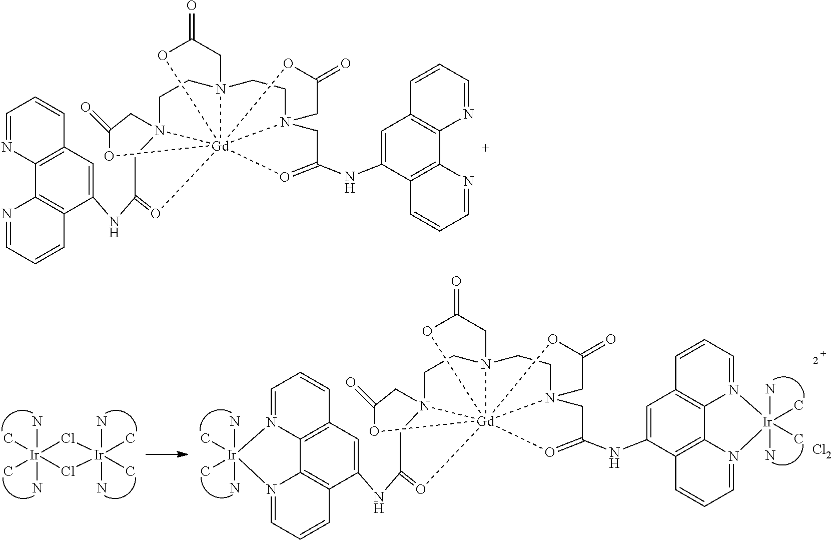

wherein each

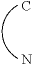

moiety is one selected form the group consisting of

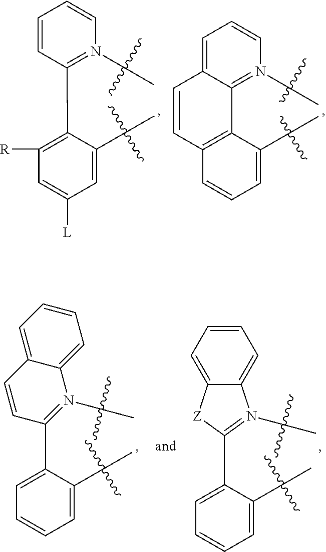

wherein R, L and Z each are defined the same as in formula I.

In some embodiments of the disclosure, under the condition that M is replaced with one selected from the group consisting of Br⁻, I⁻, NO₃⁻, and PF₆⁻, replacing M with one selected from the group consisting of Br⁻, I⁻, NO₃⁻, and PF₆⁻ includes: subjecting a product obtained from the second coordination reaction to ion exchange reaction, a reagent for the ion exchange reaction including one selected from the group consisting of NaBr, NaI, NaNO₃, and ammonium hexafluorophosphate.

In some embodiments of the disclosure, prior to the ion exchange reaction, the method further includes concentrating a product obtained from the second coordination reaction to dry to obtain a concentrated product, and redissolving the concentrated product with dichloromethane.

In some embodiments of the disclosure, the reagent for the ion exchange reaction includes one selected from the group consisting of NaBr, NaI, NaNO₃, and ammonium hexafluorophosphate, and preferably is ammonium hexafluorophosphate. In some embodiments of the disclosure, a molar ratio of the product obtained from the second coordination reaction to the reagent for the ion exchange reaction is in a range of 1:(1-1.5), and preferably 1:(1.2-1.3).

In some embodiments of the disclosure, the ion exchange reaction is performed at a temperature of 10-50° C., and preferably 25-30° C. In some embodiments, the ion exchange reaction is performed for 0.5-5 h, and preferably 2-3 h.

In some embodiments of the disclosure, the method further includes, after obtaining a probe crude product, subjecting a probe crude product to column chromatography separation and concentration in sequence, wherein an eluent for column chromatography separation is a mixed solvent of dichloromethane and methanol. In some embodiments, a volume ratio of dichloromethane to methanol in the mixed solvent is 1:1. In the present disclosure, there is no specific limitations on the concentration, and operations well known to those skilled in the art may be adopted as long as the solvent could be removed from an eluant.

The present disclosure also provides use of the multi-modality molecular imaging probe as described in above technical solutions or the multi-modality molecular imaging probe prepared by the method as described in above technical solutions in the preparation of a contrast agent for magnetic resonance imaging or the preparation of an optical probe for optical imaging.

The following will provide a clear and complete description of technical solutions of the present disclosure in conjunction with examples of the present disclosure. Obviously, the described examples are only part of examples of the present disclosure, not all of them. Based on the examples in the present disclosure, all other examples obtained by ordinary technicians in the art without creative labor fall within the scope of the present disclosure.

### EXAMPLE 1

600 mg of Gd(NO₃)₃·6H₂O, 1100 mg of a diethylenetriaminepentaacetic acid derivative, and 80 mL of methanol were added into a reactor. In the protection of nitrogen gas, a resulting solution was stirred at 50° C. for 24 hours for first coordination reaction. A resulting reaction mixture was filtered, and a resulting precipitate was collected, obtaining a gadolinium complex. MS (ESI⁺) m/z of the obtained gadolinium complex is 903.1219.

The reaction equation for the first coordination reaction was as follows:

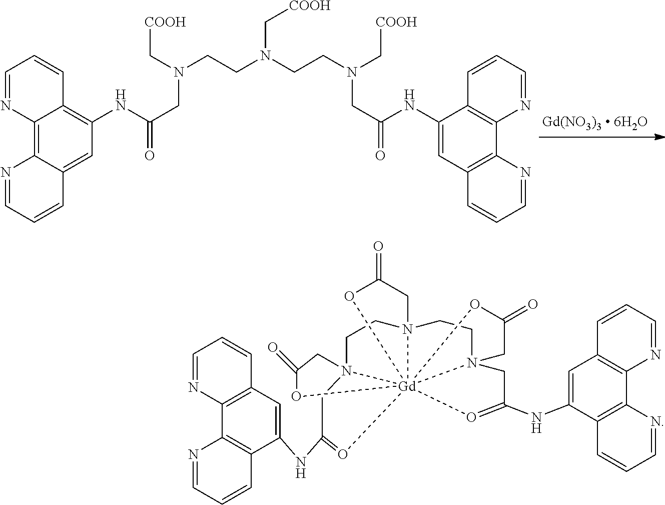

144 mg of the gadolinium complex and 206 mg of an iridium complex precursor dichlorotetrakis(2-phenylpyridine)diiridium(III) [Ir₂(ppy)₄Cl₂] were added into 50 mL of a mixed solvent of dichloromethane and methanol (with a volume ratio being 1:1). In the protection of nitrogen gas, a resulting solution was subjected to second coordination reaction at 55° C. for 24 hours, obtaining a gadolinium complex.

The reaction equation for the second coordination was as follows:

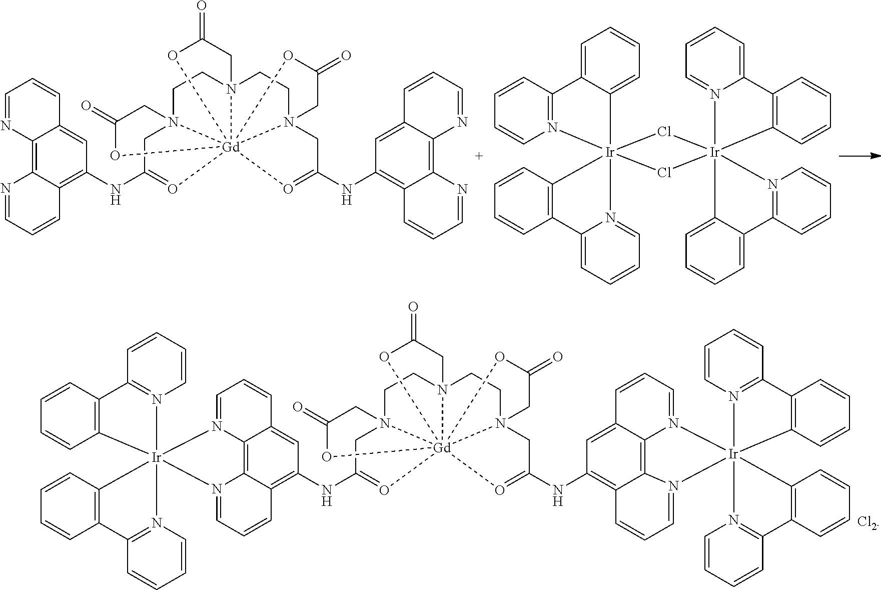

### EXAMPLE 2

In this example, the second coordination reaction was carried out according to the procedures as described in Example 1. A product obtained from the second coordination reaction was subjected to rotatory evaporation to dry, and a small amount of dichloromethane was then added thereto to dissolve it. A resulting solution was subjected to ion exchange reaction with 58 mg of ammonium hexafluorophosphate at 25° C. for 1 h, obtaining a probe crude product. The probe crude product was purified by column chromatography, in which a mixture of dichloromethane and methanol (a volume ratio being 1:1) was used as the eluent. An eluant obtained was concentrated, obtaining the multi-modality molecular imaging probe.

In this example, the reaction equation of the ion exchange reaction was as follows:

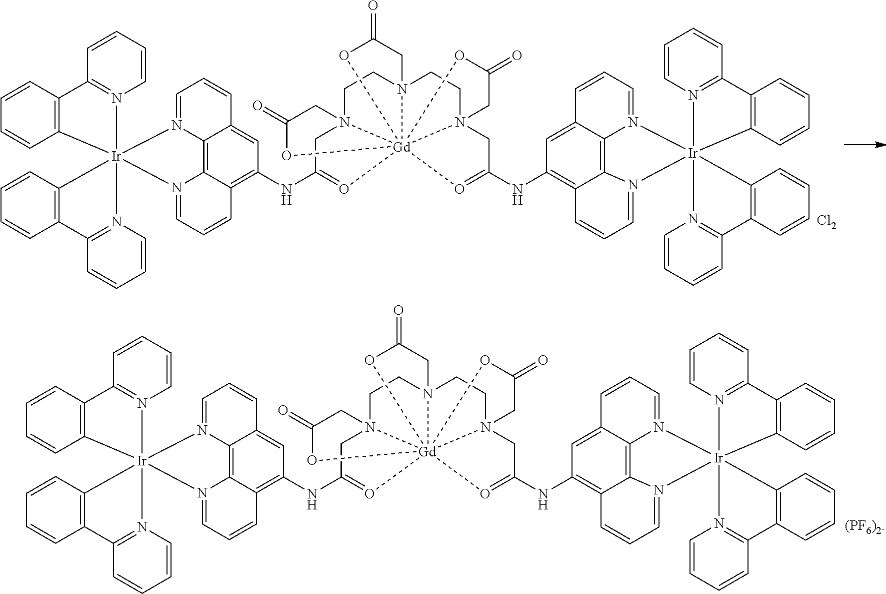

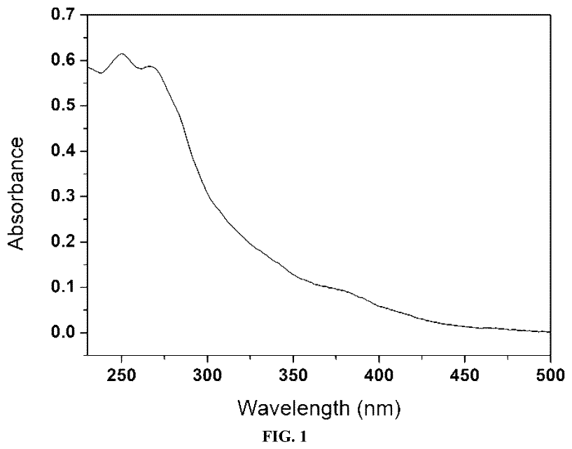

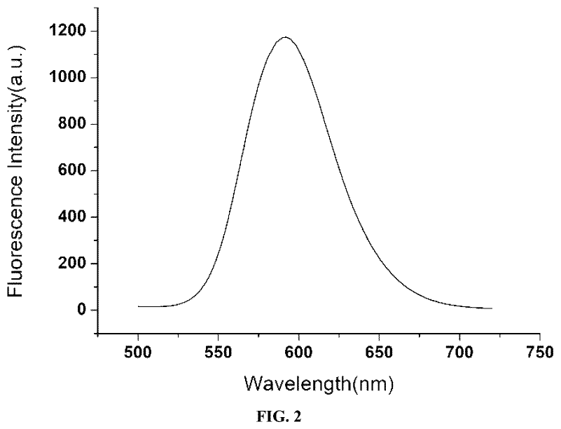

In the present disclosure, the optical properties of the multi-modality molecular imaging probe prepared in Example 2 were investigated. The UV-visible absorption spectrometry and phosphorescence emission spectrometry were conducted on an aqueous solution of the multi-modality molecular imaging probe, in which the phosphorescence emission spectrum was excited by 380 nm ultraviolet light. The test results are shown in FIGS. 1 and 2. As can be seen from FIG. 1, the multi-modality molecular imaging probe has strong absorption in the ultraviolet region (230-320 nm) and relatively weak absorption in the range of 320-450 nm. As can be seen from FIG. 2, the multi-modality molecular imaging probe exhibits phosphorescence emission in the range of 520-700 nm under excitation of 380 nm ultraviolet light, with an emission peak at around 580 nm. This UV absorption and phosphorescence emission properties lay the foundation for the probe's use in optical imaging.

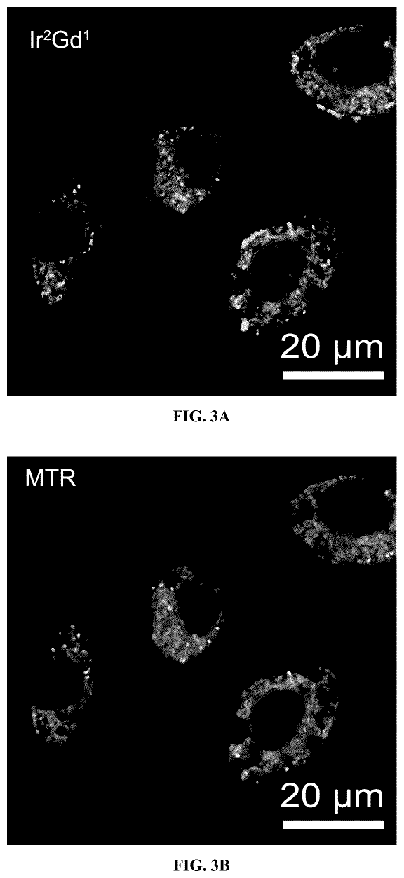

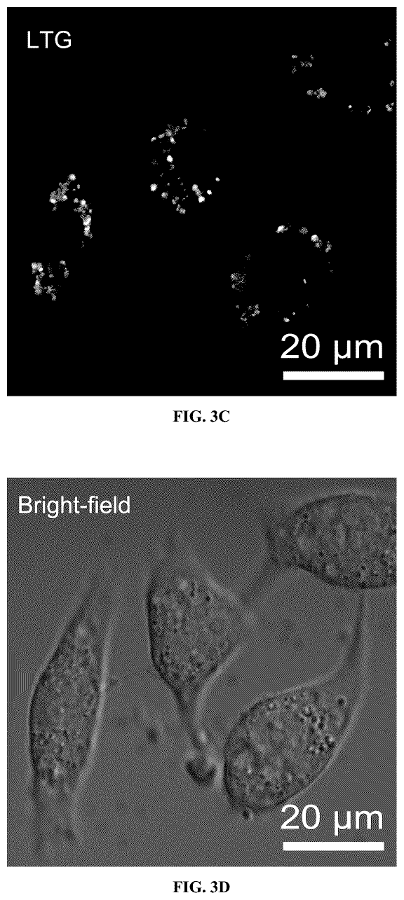

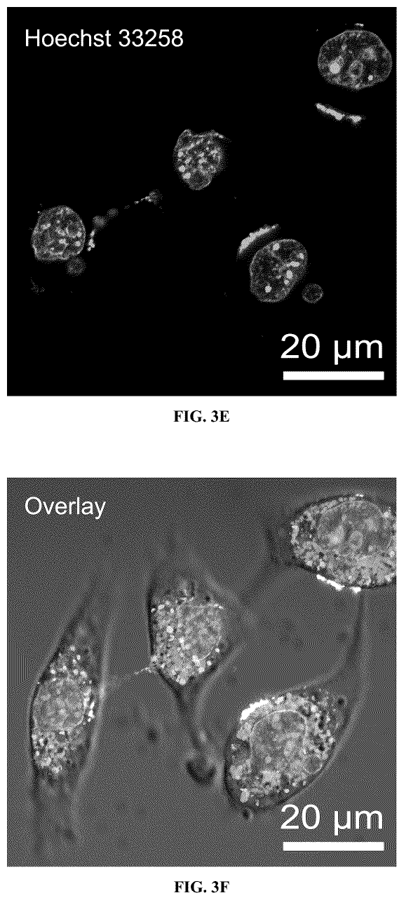

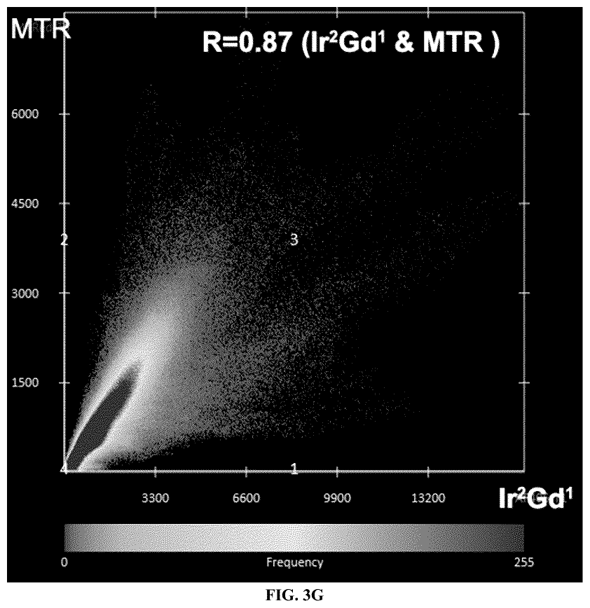

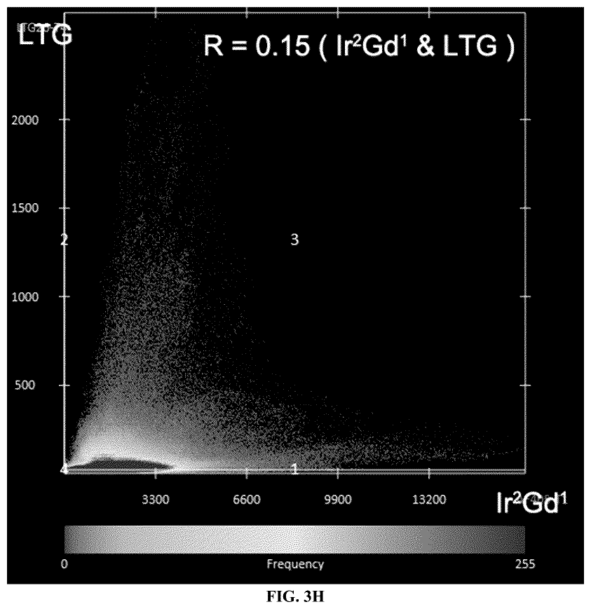

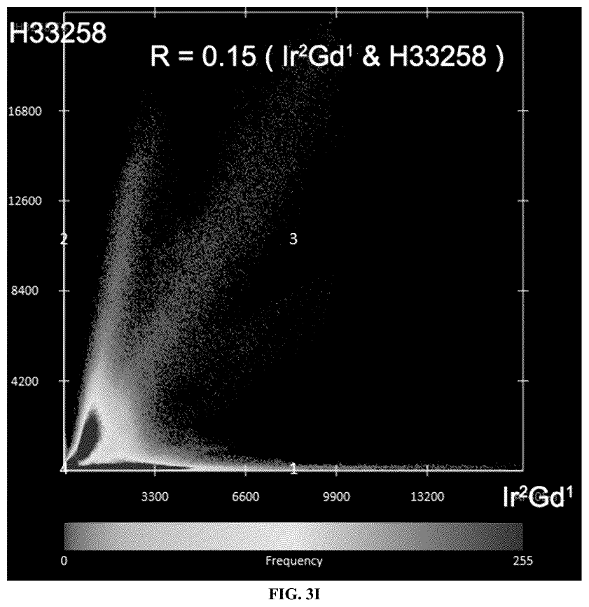

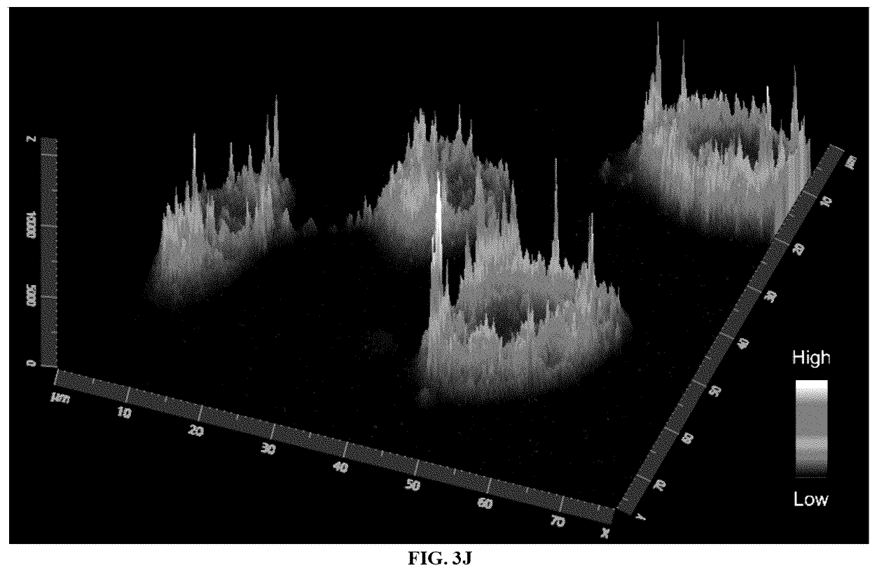

In the present disclosure, the optical imaging of living cells using a multi-modality molecular imaging probe prepared in Example 2 was investigated. The experiment process was as follows: EMT6 cells in logarithmic growth phase were seeded in a 35 mm confocal petri dish, and grew on the wall in 1640 culture medium containing 10% Fetal bovine serum at 37° C. for 24 hours. 30 μM multi-modality molecular imaging probe was then added thereto, and then the cells were incubated for 2 hours. Cell nucleus dye Hochest 33258, mitochondrial red fluorescent probe (Mito-Tracker Red, MTR), and Lysosome green fluorescent probe (Lyso-Tracker Green, LTG) were added thereto, and a resulting system was incubated for 15 minutes. A resulting mixture was washed with phosphate-buffered saline (PBS) three times, and then confocal microscopy imaging was conducted immediately. Laser scanning confocal imaging was performed on Zeiss laser Confocal microscopy LSM900. The experimental results are shown in FIGS. 3A to 3J. As can be seen from FIG. 3A, the EMT6 cells were incubated with the multi-modality molecular imaging probe (30 μM) alone for 2 h at 37° C., the phosphorescent signals localized inside the cells were observed, indicating that the inventive probe (i.e., Ir²Gd¹) could be internalized into living cells. And the phosphorescent signals presented a punctate distribution pattern, suggesting their potential localization within certain subcellular organelles. As can be seen from FIG. 3E, the bright-field image of the multi-modality molecular imaging probe-treated cell kept good shape and appeared viable. This suggests that this multi-modality molecular imaging probe could enter cells and be used for living cell imaging. As shown in FIG. 3B, when co-staining together with MTR, the signal of the inventive probe (i.e., Ir²Gd¹) highly overlaps with that of the commercial dyes of mitochondria, with Pearson's colocalization coefficient (PCC) value being 0.87 (FIG. 3G). In contrast, when co-staining together with LTG or Hochest 33258 (FIGS. 3C and 3D, respectively), there is much less overlap between the inventive probe (i.e., Ir²Gd¹) and LTG (PCC=0.15) (FIG. 3H), as well as between the inventive probe (i.e., Ir²Gd¹) and Hochest 33258 (PCC=0.15) (FIG. 3I). As shown in FIG. 3F, the overlay image of the inventive probe (i.e., Ir²Gd¹) co-stained with MTR, LTG, and Hochest 33258 suggest that the inventive probe has a very good counterstain compatibility, which is valuable for studying the uptake, bioaccumulation in living cells. The confocal 2.5 D image displays a high signal intensity (as shown in FIG. 3J), indicating a subcellular localization in the mitochondria. This indicates that the inventive probe entered mitochondria in cells and had a mitochondrial targeting property.

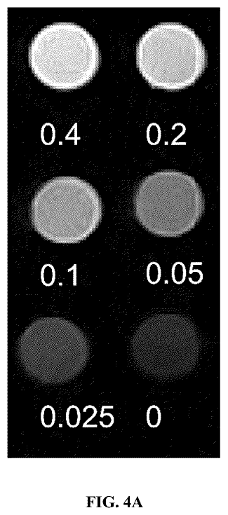

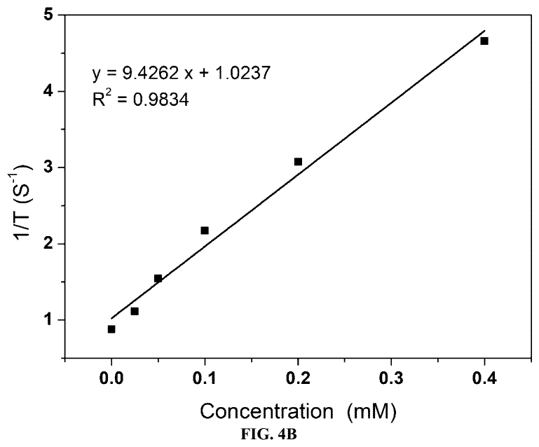

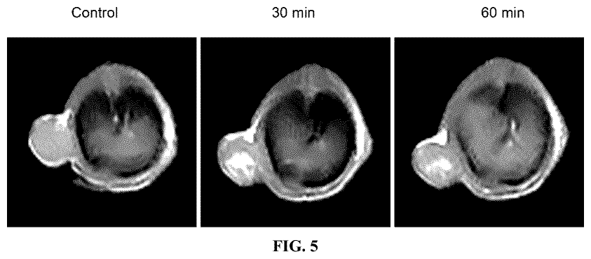

In the present disclosure, the multi-modality molecular imaging probe was tested for determining relaxation rate. A testing method was as follows: 1.5 mL of solutions (i.e., samples) respectively with a concentration of 0.4 mM, 0.2 mM, 0.1 mM, 0.05 mM, 0.025 mM, and 0 mM were prepared from the multi-modality molecular imaging probe and an aqueous solution of acetonitrile with a volume concentration of 50%. The samples were subjected to T₁ weighted imaging using an MesoMR nuclear magnetic resonance analysis and imaging system (produced by Shanghai Niumag Electronic Technology Co., Ltd., China), with a resonance frequency of 23.314 MHz, a magnetic field strength of 0.5 T, a coil diameter of 60 mm, and a magnet temperature of 32° C. The experimental results are shown in FIGS. 4A and 4B. As can be seen from FIGS. 4A and 4B, the concentration of the multi-modality molecular imaging probe is proportional to 1/T₁, that is to say, the higher probe concentration results in a brighter T₁ weighted image; and the relaxation rate is as high as 10 mM⁻¹s⁻¹, which is much higher than that of commercial gadolinium contrast agents.

In the present disclosure, in vivo magnetic resonance imaging by using the multi-modality molecular imaging probe was investigated. The experiment was conducted as follows: a 4T1 tumor-bearing mouse (about 20 g) was taken and 150 μL of 8 wt % chloral hydrate solution was administrated through intraperitoneal injection; the 4T1 tumor-bearing mouse was anesthetized and then imaged as a blank image; subsequently, the multi-modality molecular imaging probe was injected into the tumor of the experimental mouse; at two time points, i.e., 30 minutes and 1 hour after the injection, the magnetic resonance imaging was performed on the 4T1 tumor-bearing mouse by using MesoMR23-060H-I medium size nuclear magnetic resonance analysis and imaging system (produced by Suzhou Niumag Electronic Technology Co., Ltd., China), with a resonance frequency of 23.313 MHz, a magnetic field strength of 0.55 T, a coil diameter of 40 mm, and a magnet temperature of 32° C. Coronal plane and transverse plane images of the 4T1 tumor-bearing mouse were acquired by using magnetic resonance imaging software and MSE sequences. The experimental results are shown in FIG. 5. As can be seen from FIG. 5, the tumor could be observed in the transverse plane view of the 4T1 tumor-bearing mouse. By comparing images at different time points, it can be seen that the multi-modality molecular imaging probe has been injected into the tumor, and exhibits significant signal intensity on the tumor. As the post-injection time increases, the brightness at the tumor also increases. The signal strength is best at 30 minutes, and then the brightness at the tumor begins to decrease. At 60 minutes, the contrast agent has already started to be metabolized, and the signal intensity weakens.

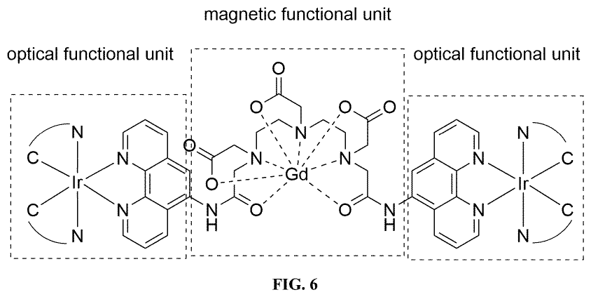

In the present disclosure, the structure formula of the multi-modality molecular imaging probe has been depicted as shown in FIG. 6, which has an ABA structure, wherein B represents a magnetic functional unit of a gadolinium complex, and A represents a phosphorescence functional unit of an iridium complex, which are reasonably integrated into the same one complex molecule. This multi-modality molecular imaging probe introduces two optical functional units of an iridium complex and one magnetic functional unit of a gadolinium complex into the same one complex molecule, which exhibits magnetic-optical dual functional properties. It therefore could be used to prepare both a contrast agent for magnetic resonance imaging and an optical probe for optical imaging.

The above are only the preferred embodiments of the present disclosure. It should be pointed out that for ordinary technicians in the art, several improvements and embellishments could be made without departing from the principles of the present disclosure. These improvements and embellishments should also be deemed as falling within the scope of the present disclosure.

What is claimed is:

1. A multi-modality molecular imaging probe, having a structure represented by formula I, 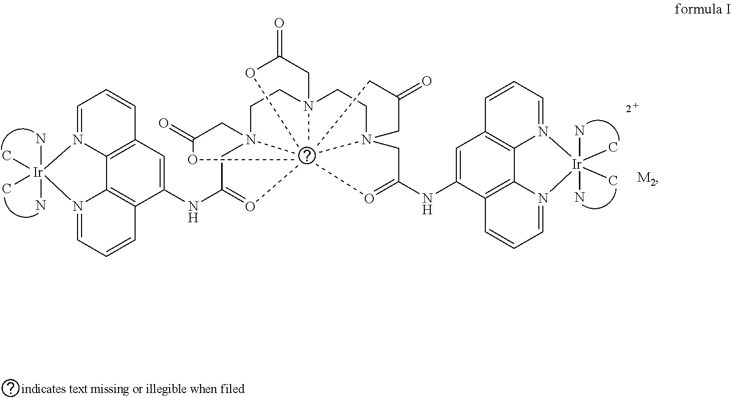
wherein in formula I, M comprises Cl⁻; and
each 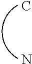
moiety is one selected from the group consisting of 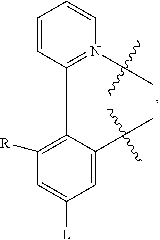 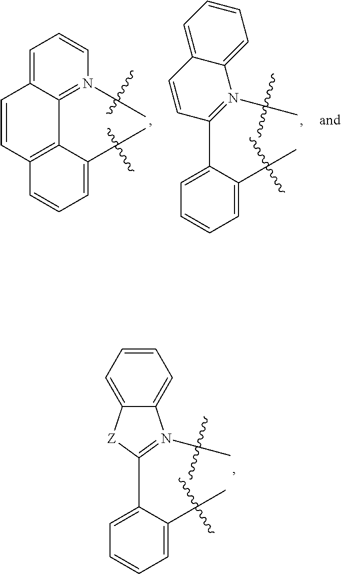
wherein
R and L are each independently selected from the group consisting of a halogen, an alkyl, and hydrogen; and
Z is selected from the group consisting of —O— and —S—.

2. The multi-modality molecular imaging probe as claimed in claim 1, wherein M is replaced with one selected from the group consisting of Br⁻, I⁻, NO₃⁻, and PF₆⁻.

3. A method for preparing the multi-modality molecular imaging probe as claimed in claim 1, comprising steps of
dissolving an inorganic gadolinium salt and a diethylenetriaminepentaacetic acid derivative, and performing first coordination reaction, to obtain a gadolinium complex; and
dissolving the gadolinium complex and an iridium complex precursor, and performing second coordination reaction, to obtain the multi-modality molecular imaging probe;
wherein the diethylenetriaminepentaacetic acid derivative has a structure represented by formula I-1, 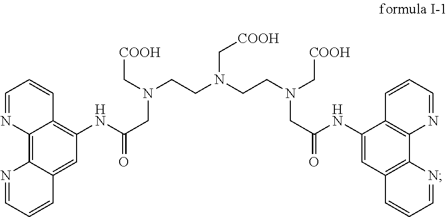 [SMILES: O=C(O)CN(CCN(CC(=O)O)CC(=O)Nc1cc2cccnc2c2ncccc12)CCN(CC(=O)O)CC(=O)Nc1cc2cccnc2c2ncccc12]
and
the iridium complex precursor has a structure represented by formula I-2, 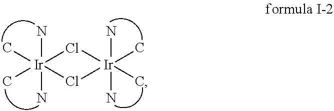
wherein in formula I-2, each 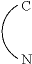
moiety is one selected from the group consisting of 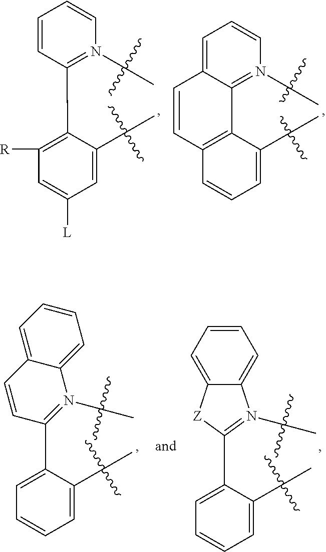
wherein
R and L are each independently selected from the group consisting of a halogen, an alkyl, and hydrogen; and
Z is selected from the group consisting of —O— and —S—.

4. The method as claimed in claim 3, wherein the inorganic gadolinium salt comprises one or more selected from the group consisting of gadolinium nitrate, gadolinium chloride, and gadolinium perchlorate.

5. The method as claimed in claim 3, wherein a ratio of an amount in moles of the inorganic gadolinium salt to a sum of amounts in moles of the inorganic gadolinium salt and the diethylenetriaminepentaacetic acid derivative is in a range of (0.2-0.8):1.

6. The method as claimed in claim 3, wherein a ratio of an amount in moles of the gadolinium complex to a sum of amounts in moles of the gadolinium complex and the iridium complex precursor is in a range of (0.3-0.7):1.

7. The method as claimed in claim 3, wherein the first coordination reaction is performed at a temperature of 45-55° C. for 23-25 h.

8. The method as claimed in claim 3, wherein the second coordination reaction is performed at a temperature of 40-60° C. for 22-26 h.

9. The method as claimed in claim 3, wherein under the condition that M is replaced with one selected from the group consisting of Br⁻, I⁻, NO₃⁻, and PF₆⁻, the method further comprises, after the second coordination reaction, subjecting a product obtained from the second coordination reaction to ion exchange reaction,
wherein a reagent for the ion exchange reaction comprises one selected from the group consisting of NaBr, NaI, NaNO₃ and ammonium hexafluorophosphate.

10. The method as claimed in claim 5, wherein the first coordination reaction is performed at a temperature of 45-55° C. for 23-25 h.

11. The method as claimed in claim 6, wherein the second coordination reaction is performed at a temperature of 40-60° C. for 22-26 h.

12. The multi-modality molecular imaging probe as claimed in claim 2, wherein the multi-modality molecular imaging probe has a structure selected from the group consisting of 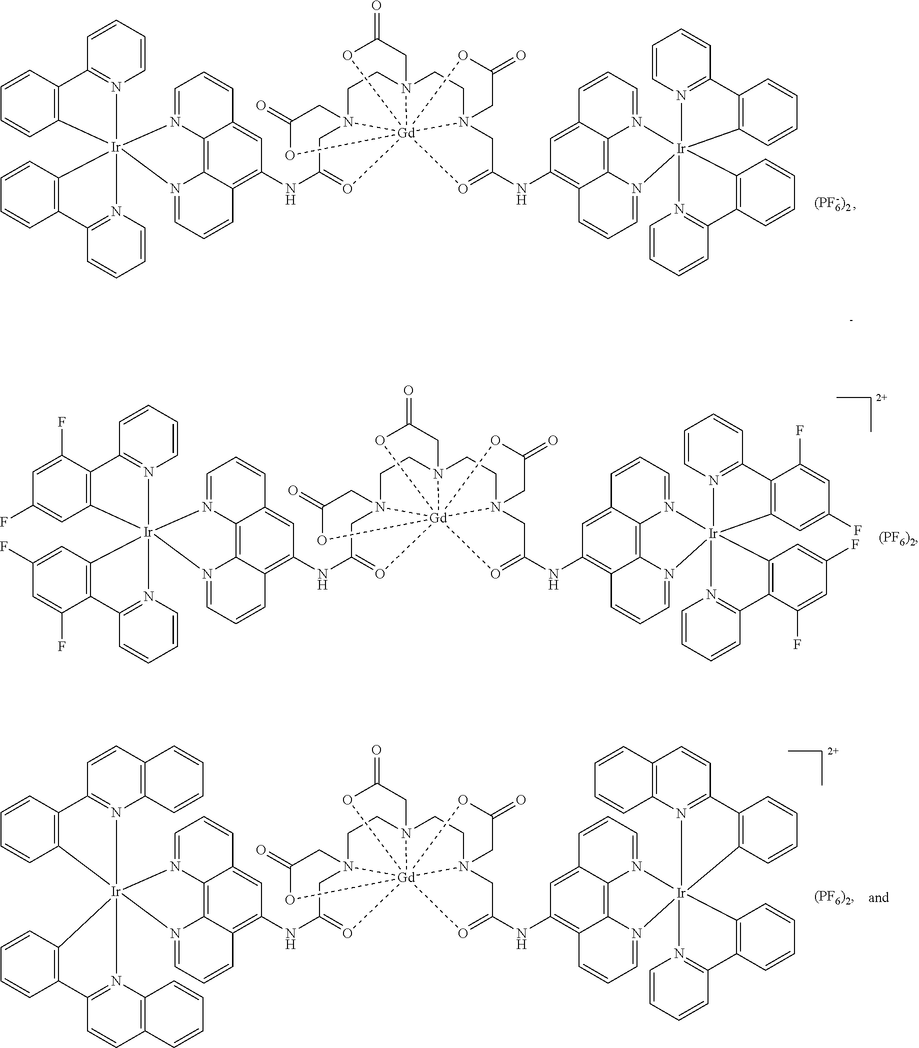 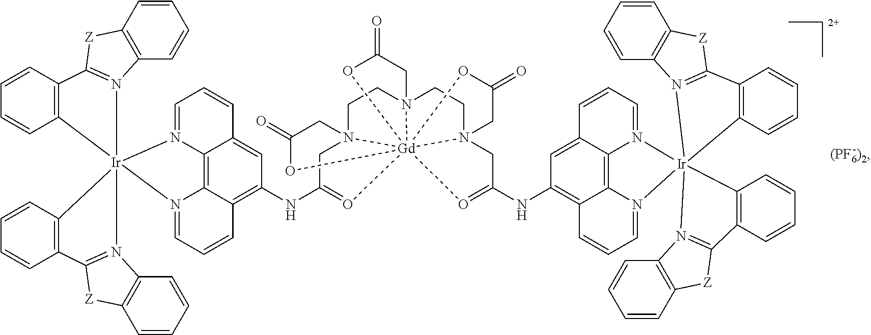
wherein Z is selected from the group consisting of —O— and —S—.

13. A method for preparing a contrast agent for magnetic resonance imaging, comprising step of
using the multi-modality molecular imaging probe as claimed in claim 1.

14. A method for preparing an optical probe for optical imaging, comprising step of
using the multi-modality molecular imaging probe as claimed in claim 1.
